(19) United States
(12) Patent Application Publication
(10) Pub. No.: US 2025/0161629 A1
(43) Pub. Date: May 22, 2025

(54) POLYOLEFIN CATHETERS

(71) Applicant: Zeus Company LLC, Orangeburg, SC (US)

(72) Inventors: Bhavya Singhi, Columbia, SC (US); John Richard Campanelli, West Columbia, SC (US)

(21) Appl. No.: 18/949,008

(22) Filed: Nov. 15, 2024

Related U.S. Application Data
(60) Provisional application No. 63/600,122, filed on Nov. 17, 2023.

(51) Int. Cl. A61M 25/00

(52) U.S. Cl. CPC A61M 25/005

(57) ABSTRACT

The present disclosure provides catheters comprising a polyolefin-based liner forming an inner surface of the catheter for at least a part of its length; a reinforcing braid disposed in and/or around the polyolefin liner for at least a part of its length; and a polyolefin-based jacket forming an outer surface of the catheter, disposed in and/or around the braid for at least a part of its length. The disclosure further provides methods of making and using such catheters.

### CROSS-REFERENCE TO RELATED APPLICATIONS

The present application claims priority to U.S. Provisional Patent Application No. 63/600,122, filed Nov. 17, 2023, the disclosure of which is incorporated herein by reference in its entirety.

### FIELD OF THE DISCLOSURE

The present application relates generally to the field of catheters, comprising two or more types of polyolefin resins and to methods and properties thereof, relating to such products.

### BACKGROUND

Vascular therapy uses minimally invasive, catheter-based procedures and specialized equipment and techniques. A conventional catheter comprises multiple layers, the innermost being a liner, which by itself typically has a wall thickness between about 0.025 mm and about 0.070 mm and an inside diameter between about 0.380 mm and about 4.300 mm. A braided layer made of metallic or polymeric material may be associated with the liner (e.g., in some embodiments, embedded in and/or enveloping the liner), and a jacket forms the outermost layer. The structural and mechanical integrity of the catheters is important for all applications. For example, when certain devices (e.g., flow diversion tubes, embolization coils, aneurysm bridging devices, scaffolding and thrombectomy devices) are passed through microcatheters in a compressed state, they exert an outward radial force causing increased pressure on the catheter. Due to the critical nature of catheter applications, the bond between the liners and jackets is extremely crucial and any delamination between the liner and jacket can lead to mechanical failure in the catheter.

Among the various materials that have been utilized as liners, e.g., within such catheter devices, the most used material is polytetrafluoroethylene (PTFE) due to its excellent chemical resistance, high temperature resistance, biocompatibility, and very low coefficient of friction/high lubricity. Some other materials which can be used as liners are polyethylene materials, especially the higher density grades, which have a significantly lower coefficient of friction than that of other commonly extruded polymers such as polypropylene. Polyethylene is also radiation-stable, so a catheter liner made from polyethylene materials may be sterilized using irradiation.

Typical materials used for catheter jackets are usually polyurethane based, such as PEBA, nylon, polyurethane, etc. The flexibility of these materials is a desirable characteristic which allows catheters to pass through vasculature that involves sharp twists and turns (e.g., cerebral vasculature and below-the-knee (BTK) applications). The nylon, PEBA or polyurethane-based jacket resins are incompatible with commonly used liner materials such as PTFE and polyethylene. To bond these liners with the jacket, the outer surface of the liner is modified by methods such as chemical etching, plasma treatment, corona discharge, etc., or with a tie-layer added to the outer surface of the liner to act as an adhesive for bonding to the jacket material. However, the innate incompatibility of the typical materials used for liners (PTFE/polyethylene) and jackets (polyurethanes/nylons) makes the bond between the two layers heavily dependent on the surface treatment or the tie-layer.

There is therefore a need for a catheter with homogeneous bonding between the liner and the jacket materials, which will eliminate the risks of delamination and critical mechanical failure.

### SUMMARY

The present disclosure provides polyolefin catheters comprising lubricious polyolefin liners, and flexible polyolefin jackets, with a reinforcement layer enclosed there between. The reinforcement layer may comprise a braid of metallic or polymeric materials.

The polyolefin liners can comprise one or more of polyethylene, polyethylene copolymers and blends thereof. The liners can have an average wall thickness of less than 0.200 mm, less than 0.100 mm (preferably less than 0.050 mm), with moderate to high machine direction orientation of the polymer.

The braided layer may be constructed using a metallic wire or a non-metallic fiber/yarn/monofilament/multifilament using polymeric materials such as polyethylene or polypropylene. This layer of the catheter provides mechanical reinforcement and torque control for improved maneuverability of the catheter.

The polyolefin jackets can comprise one or more of polyethylene, polyethylene copolymers, ethylene-propylene copolymers and blends thereof. The jackets typically have high flexibility, making them particularly suitable for use within catheters.

The present disclosure includes, without limitation, the following embodiments.

Embodiment 1: A catheter with a proximal end and a distal end defining a length, L, comprising: a polyolefin-based liner forming an inner surface of the catheter for at least a part of L; a reinforcing braid disposed in and/or around the polyolefin liner for at least a part of L; and a polyolefin-based jacket forming an outer surface of the catheter, disposed in and/or around the braid for at least a part of L.

Embodiment 2: The catheter of Embodiment 1, wherein the polyolefin-based liner forms the inner surface of the catheter for the full length of the catheter.

Embodiment 3: The catheter of Embodiment 1 or 2, wherein the polyolefin-based jacket forms the outer surface of the catheter for the full length of the catheter.

Embodiment 4: The catheter of Embodiment 1 or 3, wherein the polyolefin-based liner forms the inner surface of the catheter for less than the full length of the catheter.

Embodiment 5: The catheter of any of Embodiments 1, 2, or 4 wherein the polyolefin-based jacket forms the outer surface of the catheter for less than the full length of the catheter.

Embodiment 6: The catheter of any of Embodiments 1-5, wherein the polyolefin-based liner consists essentially of one or more polyolefins.

Embodiment 7: The catheter of any of Embodiments 1-6, wherein the polyolefin-based jacket consists essentially of one or more polyolefins.

Embodiment 8: The catheter of any of Embodiments 1-7, wherein one or both of the polyolefin-based liner and the polyolefin-based jacket comprises one or more additives.

Embodiment 9: The catheter of any of Embodiments 1-8, wherein the polyolefin-based liner has a density that differs from the density of the polyolefin-based jacket.

Embodiment 10: The catheter of any of Embodiments 1-9, wherein the polyolefin-based liner comprises one or more polyolefin resins that have a density between 0.90 g/cm³ and 0.97 g/cm³.

Embodiment 11: The catheter of any of Embodiments 1-9, wherein the polyolefin-based jacket comprises one or more polyolefin resins that have a density between 0.85 g/cm³ and 0.96 g/cm³.

Embodiment 12: The catheter of any of Embodiments 1-11, wherein the density of the polyolefin-based jacket is less than or equal to the density of the polyolefin-based liner.

Embodiment 13: The catheter of any of Embodiments 1-12, wherein the polyolefin-based liner comprises LDPE, LLDPE, HDPE, HMWPE, or UHMWPE or a blend of any two or more thereof.

Embodiment 14: The catheter of any of Embodiments 1-13 1, wherein the polyolefin-based liner has an average wall thickness of less than 0.200 mm.

Embodiment 15: The catheter of any of Embodiments 1-14, wherein the polyolefin-based jacket comprises VLDPE, ULDPE, LDPE, LLDPE, HDPE, HMWPE, or UHMWPE or a blend of any two or more thereof.

Embodiment 16: The catheter of any of Embodiments 1-15, wherein the polyolefin-based liner comprises a blend of one or more polyethylene homopolymers and one or more polyethylene copolymers.

Embodiment 17: The catheter of any of Embodiments 1-16, wherein the reinforcing braid comprises stainless steel or nitinol wires.

Embodiment 18: The catheter of any of Embodiments 1-16, wherein the reinforcing braid comprises a liquid crystal polymer, polypropylene or UHMWPE monofilament, multifilament or yarn.

Embodiment 19: The catheter of any of Embodiments 1-18, wherein the polyolefin-based jacket comprises a blend of one or more polyethylene homopolymers, one or more polyethylene copolymers, and one or more ethylene-propylene copolymers.

Embodiment 20: The catheter of any of Embodiments 1-19, comprising two or more polyolefin-based jackets, each having a different density and each disposed in and around the braid for at least less than L, wherein the two or more polyolefin-based jackets are joined together.

These and other features, aspects, and advantages of the disclosure will be apparent from a reading of the following detailed description together with the accompanying drawings, which are briefly described below. The invention includes any combination of two, three, four, or more of the above-noted embodiments as well as combinations of any two, three, four, or more features or elements set forth in this disclosure, regardless of whether such features or elements are expressly combined in a specific embodiment description herein. This disclosure is intended to be read holistically such that any separable features or elements of the disclosed invention, in any of its various aspects and embodiments, should be viewed as intended to be combinable unless the context clearly dictates otherwise. Other aspects and advantages of the present invention will become apparent from the following.

### BRIEF DESCRIPTION OF THE DRAWINGS

To provide an understanding of the embodiments of the invention, reference is made to the appended drawings, which are not necessarily drawn to scale, and in which reference numerals refer to components of exemplary embodiments of the invention. The drawings are exemplary only and should not be construed as limiting the invention. FIG. 1 is a general schematic of a typical catheter, with relevant parameters, and an expanded schematic of one cross-sectional end face of a typical catheter.

Having thus described the disclosure in the foregoing general terms, reference will now be made to the accompanying drawings, which are not necessarily drawn to scale, and wherein:

FIG. 1 is a schematic depiction of a typical, but non-limiting catheter according to certain embodiments of the present disclosure;

FIG. 2 is a schematic of an IDTE test track employed in certain examples of the present disclosure;

FIG. 3 is a plot of density vs. Shore D hardness values for various non-limiting plaque samples according to certain embodiments of the present disclosure;

FIG. 4 is a plot of flexural stress up to 1% strain values for various non-limiting plaque samples according to certain embodiments of the present disclosure;

FIG. 5 is a plot of data on pull resistance for various non-limiting catheters according to certain embodiments of the present disclosure;

FIG. 6 is a typical, but non-limiting curve of catheter pull resistance according to certain embodiments of the present disclosure;

FIG. 7 is a plot of data on lubricity for various non-limiting catheters according to certain embodiments of the present disclosure;

FIG. 8 is a typical, but non-limiting curve of catheter lubricity according to certain embodiments of the present disclosure;

FIG. 9 is a cross-sectional image of a catheter assembly according to Example 4A (250° C.); and

FIG. 10 is a cross-sectional image of a catheter assembly according to Comparative Example 4.

### DETAILED DESCRIPTION

The present invention now will be described more fully hereinafter. This invention may, however, be embodied in many different forms and should not be construed as limited to the embodiments set forth herein; rather, these embodiments are provided so that this disclosure will be thorough and complete, and will fully convey the scope of the invention to those skilled in the art. As used in this specification and the claims, the singular forms “a,” “an,” and “the” include plural referents unless the context clearly dictates otherwise.

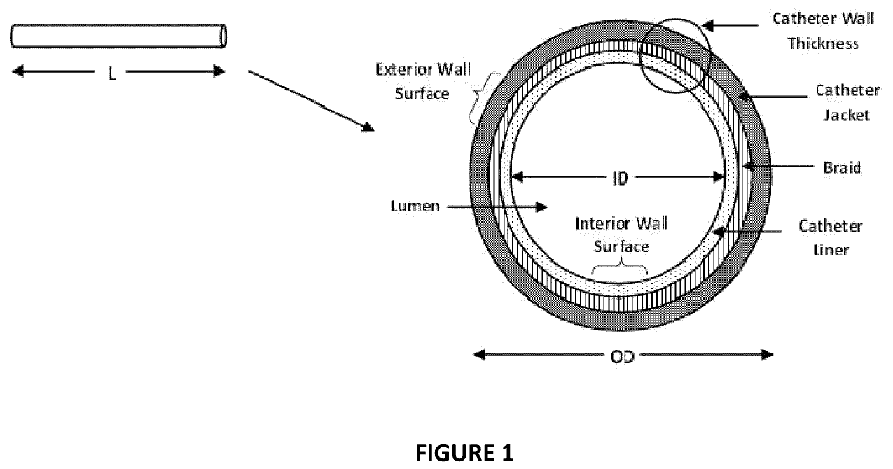

A general schematic of a catheter is shown in FIG. 1. The catheter is generally cylindrical in shape. “L” indicates the length of the catheter as produced, which can be processed, e.g., cut, to provide catheters of desired length “1” (not shown). The expanded region at the right of FIG. 1 is a cross-sectional view of the catheter. As shown, the “lumen” is an interior region of the catheter, i.e., an open channel/cavity (through which, e.g., a catheter device may be passed). The inner diameter of the catheter, shown as “ID” is the average distance from a point on the interior wall of the catheter liner to the opposite/farthest point on the interior wall of the catheter liner. The outer diameter of the catheter, shown as “OD” is the average distance from a point on the outer wall of the catheter jacket through the lumen of the catheter to the opposite/farthest point on the outer wall of the catheter jacket. As such, half of the difference between the OD value and the ID value provides the average wall thickness of the catheter. A representative “catheter wall thickness,” “interior wall surface,” and “exterior wall surface” of the catheter are shown in FIG. 1. A conventional three-layer catheter comprises a liner as the innermost layer. A braided layer envelopes the liner, and a jacket encloses the braided envelope, as shown in FIG. 1. The braided layer is optional and may or may not be incorporated into the final catheter, or incorporated along a partial length of the catheter, dependent upon the desired application of the said catheter.

The present disclosure provides lubricious and flexible polyolefin-based catheters produced by various polymer processing methods, with different physical, mechanical, thermal, and structural properties as outlined more fully herein below. The disclosure further relates to a catheter with a thin wall polyethylene liner that includes the features of having a wall of a thickness of less than about 0.100 mm, preferably less than about 0.050 mm and, more preferably less than 0.020 mm. The disclosure also provides polyolefin-based catheters which do not require an additional tie layer or surface modification of the liners and/or jackets for enhancing the bond between the liner and the jacket, as is the case with conventional catheters. Additionally, the polyolefin catheters of the current invention can have lower weight and density compared to traditional polytetrafluoroethylene (“PTFE”) and polyether-block-amide (“PEBA”)/nylon/polyurethane (“PU”)-based catheters. A detailed description of each of the layers used to make the polyolefin catheter of the present invention is provided below.

The innermost layer of the catheter, shown in FIG. 1 as “Catheter Liner” and hereinafter referred to as the “liner” is preferably made using one or more grades of polyolefin resins, including, but not limited to, very low density polyethylene (“VLDPE”), ultra low density polyethylene (“ULDPE”), low density polyethylene (“LDPE”), linear low density polyethylene (“LLDPE”), medium density polyethylene (“MDPE”), high-density polyethylene (“HDPE”), high molecular weight polyethylene (“HMWPE”), ultra high molecular weight polyethylene (“UHMWPE”), modified and/or grafted polyethylene, and combinations, derivatives, copolymers, and blends thereof. These terms are generally known in the art and one of skill in the art will be aware of suitable resins falling within the scope of each type and grade referenced above.

The amount and grade of polyolefin resins used in blend formulations can vary to obtain liners with different thermal, mechanical, and structural properties. For example, the addition of modified and/or grafted polyethylene resins such as maleic anhydride grafted LLDPE or maleic anhydride grafted HDPE in some embodiments of the liner can increase the hydrophilicity of the inner wall surface of the liner, making it more suitable for applications requiring high lubricity of a wetted inner wall surface of a catheter liner. The inner surface of a hydrophilic liner can be wetted by water or saline to improve the overall lubricity of the inner surface by providing a tribological layer that lowers the coefficient of friction (COF). Some examples of modified polyolefin resins which can be used in the formulations of the invention include maleic anhydride modified polyethylene, ethylene vinyl acetate, ethylene methyl acrylate, ethylene acrylic acid, ethylene methacrylic acid, ethylene-acrylic ester-maleic anhydride terpolymer, etc.

The thickness of the polyolefin-based liner is not particularly limited. However, in some embodiments, thin-walled polyolefin-based liners are particularly desirable. For example, in some embodiments, the polyolefin-based liner can have an average wall thickness of less than 0.100 mm or less than 0.050 mm. The polyolefin-based liner can, in some embodiments, have moderate to high machine-direction orientation of the polymer.

The polyolefin-based liner can extend the full length “L” of the catheter in some embodiments. In other embodiments, the polyolefin-based liner extends only a portion of the full length “L”, e.g., about 50% of L or more, about 60% of L or more, about 70% of L or more, about 80% of L or more, about 90% of L or more, or about 95% of L or more. The length-wise positioning of the polyolefin-based liner with respect to other catheter components can vary where it extends only a portion of the full length “L” of the catheter. In some embodiments, the polyolefin-based liner extends from a distal end toward the proximal end of the catheter (such that it is present at the distal end but not at the proximal end); in some embodiments, the polyolefin-based liner extends from a proximal end toward the distal end of the catheter (such that it is present at the proximal end but not at the distal end); in some embodiments, it is positioned toward the middle of the length L of the catheter (such that it may not be present at either the proximal or distal end of the catheter).

The liner can, in some embodiments, be associated with a reinforcing component or reinforcing layer, e.g., a “Braid” as depicted in FIG. 1. For example, the reinforcement component or layer can, in some embodiments, be positioned between at least a portion of the outer surface of the liner and the inner surface of the jacket. In some embodiments, a polyolefin fiber/yarn/monofilament/multifilament/ribbon/tape produced, e.g., by an extrusion or spinning process, can be wrapped, braided, plaited, entwined, coiled, woven, twisted, knitted, or wound, on a metallic or non-metallic mandrel/rod in a tubular structure. The tubular structure can then be compression molded/formed using a heat shrink cover to form into a tubular liner.

In some embodiments, a braided layer may be incorporated between the liner and the jacket for enhanced torque control and mechanical properties (e.g., as compared with catheters not comprising the braided layer). The braid can be constructed using metallic materials such as stainless-steel wire or polymeric fiber/yarn/monofilament/multifilament using one or more grades of polyolefin materials such as polyethylene or polypropylene.

In some embodiments, an oriented fiber/filament/yarn made of UHMWPE may be used for the braided layer. Highly drawn and oriented UHMWPE filaments can provide high hoop strength and mechanical integrity to the catheter. Due to the compatibility of UHMWPE braid with the polyethylene liners and polyolefin jackets used in this invention, this three-layered structure can fuse and form a single-layer homogeneous catheter after the reflow process.

In some embodiments, a metallic or non-metallic hypotube may be used in place of a braid as a reinforcing layer between the polyolefin liner and a polyolefin jacket. The polyolefin liner may have better bonding to the hypotube as compared to surface-treated PTFE liners. Additionally, the polyolefin liner will have a better bond with the polyolefin jacket as compared to surface-treated PTFE liners with PEBA, nylon or polyurethane-based jackets.

The outermost layer of the catheter, shown in FIG. 1 as “Catheter Jacket”) and herein referred to as the “jacket,” is generally made using one or more grades of polyolefins such as VLDPE, ULDPE, LDPE, LLDPE, MDPE, HDPE, HMWPE, UHMWPE, PP, polyolefin elastomers (POEs), polyolefin plastomers (POPs), modified and/or grafted polyethylene. These resins may be used as a blend in different weight percentages to alter physical, mechanical, thermal, and structural properties of the jacket as well as the catheter. In some embodiments, the jacket comprises an elastomeric component (e.g., such that, in some embodiments, it may be associated with the catheter in an expanded form and heat shrunk to form the catheter). In some embodiments, polyolefin jackets of different compositions and/or different properties (e.g., different densities) may be used in multiple segments along the catheter shaft to provide variable flexibility/torqueability along the length of the catheter.

The polyolefin tubes used as liners and jackets in this invention may be processed using a typical melt processing apparatus and under typical melt processing conditions. The liners can also be extruded using a ram or a paste extruder with or without a lubricant. Alternately, the liners may be produced by other polymer processing methods such as, but not limited to, dip-coating, powder-coating, dispersion coating, solvent casting, spinning, electroplating, compression molding, etc.

In some embodiments, the polyolefin liners can be manufactured by extruding/coating/molding/braiding or otherwise placing the polymer over a metallic or non-metallic wire or mandrel. Mandrels suitable for use can be made from stainless steel (“SS”), nitinol, PTFE, or polyether ether ketone (“PEEK”), for example. The surface of the mandrel can be smooth or textured. A reinforcement layer and jacket can be positioned over the polyolefin liner and reflowed with a suitable heat shrinkable forming sleeve. The catheter shaft can then be removed from the mandrel by sliding it off, or by first elongating the mandrel and removing it.

In some embodiments, to produce a jacket, the resins can be blended in a single-screw or twin-screw apparatus using typical melt processing conditions and then pelletized for tubing extrusion. The resins may also be mixed as a salt and pepper blend into the hopper of a single-screw extruder and melt extruded into tubing directly.

In some embodiments, the catheters can be manufactured by coextruding the jacket over the braided polyolefin liner. In some embodiments, an extruded polyethylene liner can be braided with a metallic or non-metallic wire/filament in a continuous or semi-continuous manner, and the polyolefin jacket can be extrusion coated on top of the braided liner.

In some embodiments, a polyethylene liner can be extruded, then braided with a metallic or non-metallic wire/filament and then extrusion coated with a polyolefin jacket on top of the braided liner structure in a continuous manner.

In some embodiments, the elastomeric polyolefin tubing for the jacket may be expanded prior to construction and used as a heat shrink material by shrinking it onto the remainder of the components.

It is to be understood that the individual components of the catheters of the disclosure may, in some embodiments, contain additives such as antioxidants, antimicrobials, processing aids, slip aids and/or colorants, as well as other particulates designed to impart specific properties to the tubes such as radiopacity. In other embodiments, they consist or consist essentially of the referenced resin or resins.

For building catheters using the liners and jackets of this disclosure, instruments such as the Beahm 810A vertical laminator can be used. The polyolefin liners of this invention can be bonded with the polyolefin jackets using heat shrink tubing typically made from PEBA, Nylon, FEP, PET, PTFE, etc. The polyolefin tubes of the invention may also be expanded and shrunk over the braided liner to form the jacket of the polyolefin catheter. The polyolefin liners can also be stretched with or without heat to reduce the wall thickness and then used to build catheters. The degree of stretching of the liners can affect some physical, mechanical, or thermal properties of the built catheters, such as lubricity, tenacity, modulus, etc. Typically, increasing the degree of stretching increases the axial orientation of the polymer, thereby increasing the modulus and tensile strength.

Polyolefin liners can also be braided with metallic or non-metallic wires/filaments/yarns before lamination with the polyolefin catheter jackets using braiding machines such as the Steeger Medical Braider. These types of braiding machines can be used to braid with different patterns, pick count, length, etc. using wires/filaments/yarns of various profiles and sizes. The change in braid pattern and pick count can affect the overall properties of the catheter, such as hoop strength, flexibility, torque response, etc.

### Experimental and Evaluation Methods

Embodiments of the present disclosure are more fully illustrated by the following examples, which are set forth to illustrate aspects of the present disclosure and are not to be construed as limiting thereof. Unless otherwise noted, all parts and percentages are by weight.

One non-limiting method for construction of the catheter assemblies is carried out in the following manner. A liner as described herein is loaded onto a mandrel and the reinforcement layer is optionally introduced, e.g., the liner is braided or coiled with specified wire, for example, using a Steeger 16 carrier as braider and a Rothgreaves unit as coil winder. After the reinforcement layer is applied, the distal end can be trimmed and covered with a heat shrink tube (e.g., a PET heat shrink tube) to secure the optional reinforcement layer, e.g., braid or coil. The jacket tubing is then loaded onto the assembly, in some embodiments so that each durometer covers approximately half of the 3 ft long shaft. The assembly is then covered with another heat shrink material (e.g., FEP) and laminated, e.g., on a Machine Solutions 4-up, 200 cm long vertical laminator. Suitable laminator run conditions include, but are not limited to, a 250° C. nozzle temperature with a speed of 1.2 mm/s and air flow rate of 40 LPM. The shrink tube is removed after cooling and the distal tip rounded for ease of tracking. The mandrel was then removed by stretching it, and the catheter shaft can be optionally sized, e.g., cut to the desired length (e.g., 36 inches overall length) to give the catheter assembly.

Several test methods can be used to evaluate the polyolefin catheters of this disclosure.

Built catheters and catheter components such as liners and jackets can be tested using an interventional device testing equipment such as the IDTE3000 from MSI which can measure and record device performance features such as push force, flexibility, torqueability, etc. Multiple test cycles can be performed on the same sample to measure change in torque/push force due to potential abrasions on the inside surface from repeated testing. The IDTE 3000 features a temperature-controlled water bath, adjustable pegboard tray that is submergible, proximal pneumatic roller system, proximal torque motor, and a dedicated PC that controls all operation.

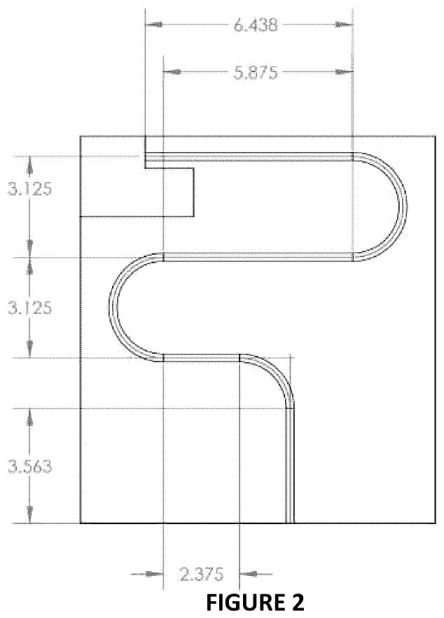

Testing is performed on the IDTE using the proximal pneumatic roller system. A standard setup for trackability consists of a product support tray, the proximal roller system, and a test track in an S-configuration (see FIG. 2) secured to the adjustable pegboard tray, wherein the pegboard tray is submerged in the water bath at 37° C. The proximal end of the test specimen is supported by the product tray, and the distal end feeds through the roller system and into the PFA tubing of the test track. The numbers shown in FIG. 2 refer to representative sizes (in inches), which are exemplary only and are not understood to be limiting of the setup. A further test is tracking a guidewire through a lumen of the catheter to determine resistance to delivering a therapy through the catheter. In this case, the catheter is secured onto the test track and the proximal end is secured by a C-clamp on the roller system. A guidewire is laid flat on the product tray and the distal tip is fed through the roller system and into the lumen of the catheter until the tip is correctly positioned within the test track. This test method can be used to evaluate performance of a catheter liner by measuring the force required to insert and retract the guidewire through the catheter.

The following parameters are used to test trackability on the track shown in FIG. 2:
Roller pressure: 10-20 psi adjusted as necessary to prevent slippage or collapse of the test catheter wall at the proximal end.
Load cell: 3 Kg.
Water temperature: 37° C.
Test speed: 100-150 cm/min

For catheter trackability, a 20 cm total test path is used extending from the proximal to the distal end of a PFA tube configured as the track according to the layout shown in FIG. 2. For guidewire trackability, a ViperWIRE Advance VPR-GW-14 Guidewire (0.014″) is used with the test catheter configured as the track according to the layout shown in FIG. 2.

The maximum force measured during the advancing portion of the test is recorded. The work performed to advance the catheter or guide wire is calculated using software such as OriginPro® to integrate the area beneath the plot of force (F) vs distance (x). The formula for calculating Work of Insertion (W) is:

W=∫_{A}^{B}F(x)dx, where A=initial position of the catheter and B=final position of the catheter.

ASTM D2240 is used to measure durometers on the A and D scales for plaques made using Pebax, polyolefin and compounded blends as described in the examples.

A TA instruments Q800 DMA with a 3-point bend fixture is used to determine the flexural properties of the polyethylene, polyolefin and PEBA plaques (ASTM D5023). The test is operated at room temperature using a span length of 15 mm with a strain ramp of 1%/min up to 5% strain. The data is used to compare the flexural stress of different catheter materials. The test can also be performed on tubing and catheter samples. The DMA can also be used to measure additional thermo-mechanical or visco-elastic properties at other temperatures and conditions.

The density of the compounded polyolefin resin blends used for making the catheters is calculated using the equation below, wherein,

| | |
|---|---|
| mₐ = Mass of resin A | m_{b} = Mass of resin B |
| pₐ = Density of resin A | p_{b} = Density of resin B |

p_{c} = Calculated density of blend of resins A and B

$${ρ}_{c} = \frac{{m}_{a} + {m}_{b}}{\frac{{m}_{a}}{{ρ}_{a}} + \frac{{m}_{b}}{{ρ}_{b}}}$$

A mechanical tester such as an Instron 5965 dual column can be used to determine the tensile properties of the individual layers, as well as the built catheters. The test can be performed at room temperature (23° C.) and an environmental chamber can be used to measure tensile properties at an elevated temperature. The mechanical tester can also be used to measure the peel resistance of the built catheters of this invention as well as traditional catheters from comparative examples. The test method used to evaluate bondability is a modification of ASTM D1876 (T-Peel Test), wherein the laminated test sample of the ASTM standard is a bonded catheter tube split in half. The liner is gripped on one side and the jacket is gripped on the other. The test is performed at room temperature (23° C.) at a rate of 76.2 mm/min using a 100 N load cell set to a 25.4 mm gage length.

An Instron 5965 dual column mechanical tester is used for a three-point bend test as per ASTM D790-07 on tubing samples with a 10N load-cell, support span of 1.5 in and a test rate of 0.04 in/min. The data collected is used to assess flexural stress and modulus in relation to increasing flexural strain.

Samples are tested for contact angle using a Tantec Inc. CAM-PLUS Contact Angle Meter using half-angle method per ASTM D7334.

The Harland FTS7000 measures surface friction by drawing a test sample between two 60D silicone rubber pads clamped at a programmable force and recording the pull resistance (frictional force). The frictional force (g) is plotted with the sample position over the test duration. The coefficient of friction is calculated using the recorded Frictional Force and applied Clamp Force, Ff/Fc. Samples were evaluated while submerged in a 37C DI water bath with an applied clamp force of 500 g. The test velocity is 5 mm/s over a 300 mm distance. There is a 10 sec hydration pause at the start of the test and a 5 sec pause time between each of the 5 test cycles.

Microscopic images of the catheters are taken using an optical microscope to observe the interface between the different layers of the catheters. The cross-section of catheters are viewed using a Starrett MVR300 vision system and the MetLogix software was used to capture images at 2× magnification. The microscope can also be used to check for abrasion on the inside surface of the liners and/or catheters, after several cycles of simulated use, such as sliding a guide wire through the catheter repeatedly or measuring the push force on the same sample multiple times.

Hoop strength or burst pressure of the individual tubing components as well as the built catheters can be measured using standard test methods such as the ASTM D1599-18.

A standard gas pycnometer can be used to measure the density of the individual tubing samples as well as the built catheters.

Standard or modified taber test for abrasion maybe used to compare abrasion resistance of liners and catheters of this invention and typical PTFE liners and catheters.

A TA instruments Discovery Hybrid Rheometer (DHR-3) rheometer with the tribo-rheometer accessory can be used to determine the tribological properties of the polyolefin tubing used as liners. The main property of interest during this test would be the coefficient of friction (COF). The tests can be performed at room temperature and elevated temperatures, with or without a tribological layer (such as saline). The tribo-rheometer can also be used to analyze abrasion resistance on the inside surface of the liners and/or catheters, after several cycles of simulated use, such as sliding a guide wire through the catheter repeatedly or measuring the push force on the same sample multiple times.

A Dynisco LMI4003 Melt Indexer can be used to measure the melt flow index (MFI) of the resin blends used for making the different layers of the current invention disclosure.

A Mitutoyo Surftest SV-400 Profilometer can be used to measure the surface roughness of the outer surface or the inside surface of the liners and/or catheters, after several cycles of simulated use, such as sliding a guide wire through the catheter multiple times. Other laser profilometer methods may also be used to measure surface roughness of samples.

### EXAMPLES

Various polyolefin resin blends, as listed in Table 1, were compounded in a Leistritz 25 mm twin-screw extruder with different formulation ratios and pelletized for use in other melt processing. The compounding temperatures used were between 100 to 220° C. across the multiple heat zones in the barrel and die of the extruder. The compositions used for further processing and testing have been described below in detail in addition to their processing methods.

TABLE 1
Resins used for Polyolefin Catheter Compositions

| Resin ID | Resin Grade | MFI (g/10 min) | Density (g/cm³) |
|---|---|---|---|
| LDPE 1 | Engage 8842 | 1.0 | 0.86 |
| LDPE 2 | Attane 4201 | 1.0 | 0.91 |
| LDPE 3 | Attane 4404 | 4.0 | 0.90 |
| LDPE 4 | Flexomer DFDA 1137 | 1.0 | 0.90 |
| LDPE 5 | Bormed ™ LE6600 | 1.5 | 0.92 |
| HDPE 1 | Bormed ™ HE7541 | 4.0 | 0.95 |
| HDPE 2 | Orevac ® 18507 | 5.0 | 0.95 |
| UHMWPE | Mipelon ™ PM200 | — | 0.94 |

### Plaque Examples

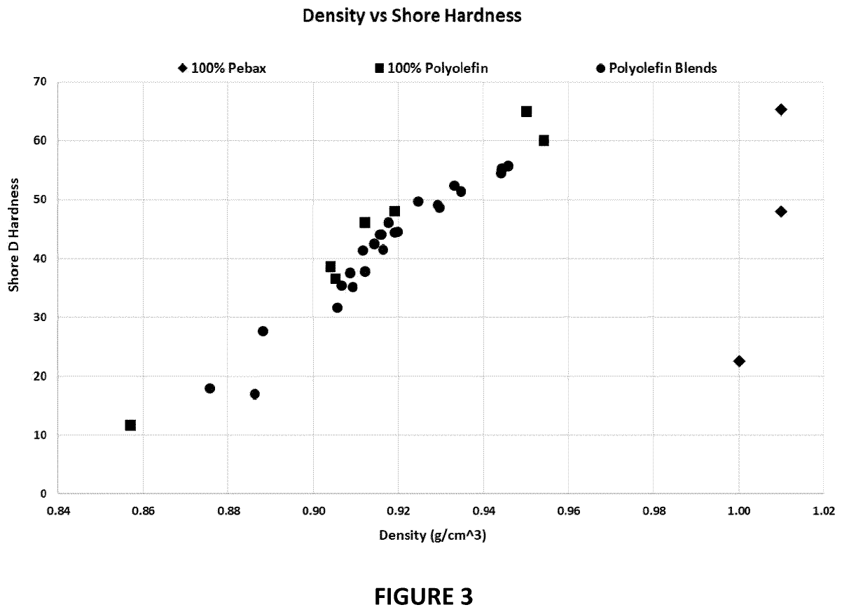

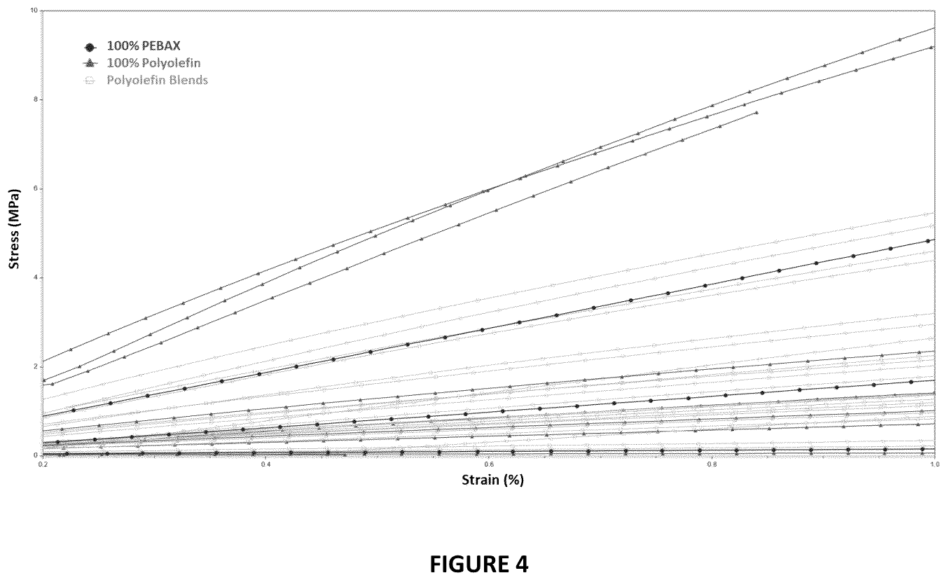

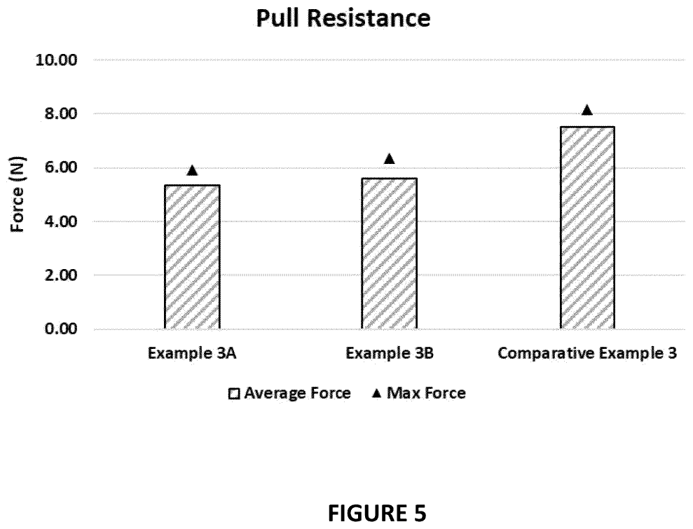

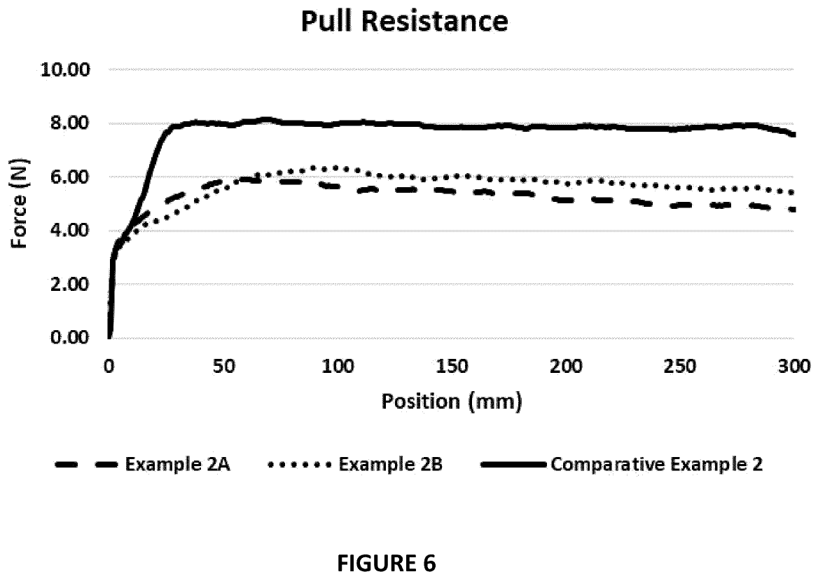

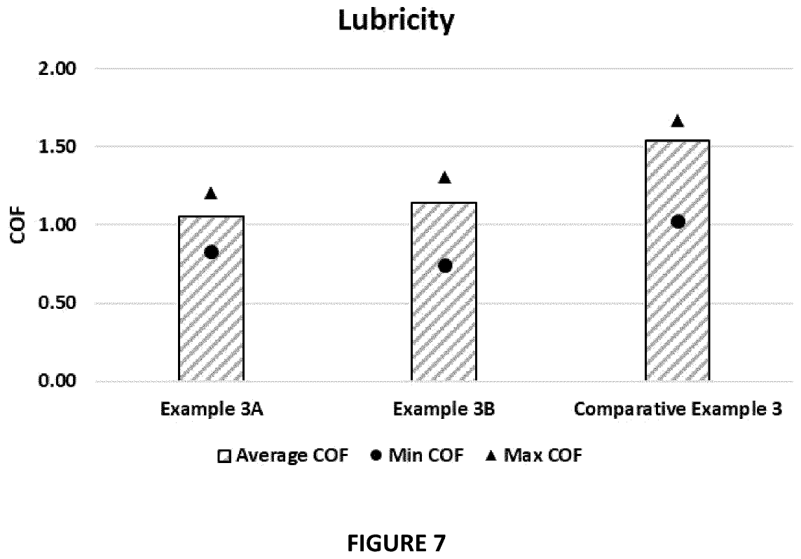

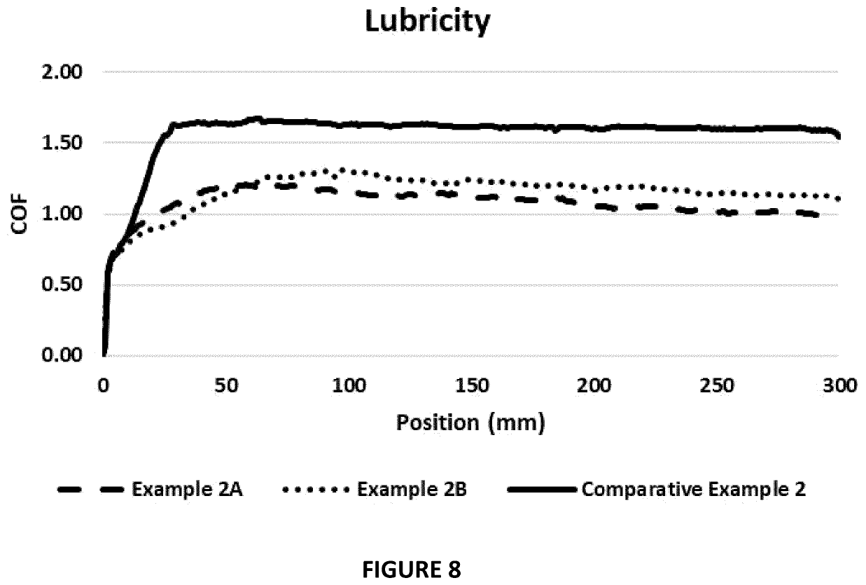

Plaques of various PEBA and polyolefin resins, and their compounded blends, were prepared in a Carver Melt Press using a stainless-steel mold with a thickness of about 3.3 mm at a temperature 30 to 60° C. greater than the melt temperatures of the resins. The plaques were subjected to shore hardness testing for durometers A and D using standard test methods and flexural stress was measured on a DMA with a 3-point bend fixture. The results of some of the plaque formulations are listed in Table 2 as well as shown in FIGS. 3-4.

TABLE 2
Durometer and Flexural Stress Results
for some of the Plaque Samples

| | | | | Flex- | Flex- |
|---|---|---|---|---|---|
| | | | | ural | ural |
| | | | | Stress | Stress |
| | | | | @ | @ |
| Com- | | | | 1% | 3% |
| position | | Shore | Shore | Strain | Strain |
| # | Plaque Composition | A | D | (MPa) | (MPa) |
| 1 | 100% Pebax 2533 | 78.7 | 22.6 | 0.17 | 0.44 |
| 2 | 100% Pebax 5533 | 97.1 | 47.9 | 1.72 | 5.15 |
| 3 | 100% Pebax 7233 | 98.2 | 65.3 | 5.18 | — |
| 4 | 100% LE6600 | 97.8 | 48.1 | 2.44 | 5.76 |
| 5 | 100% HE7541 | 91.5 | 60.1 | 9.22 | — |
| 6 | 100% Engage 8842 | 55.6 | 11.7 | 0.07 | 0.15 |
| 7 | 100% Attane 4201 | 96.6 | 44.3 | 1.07 | 3.79 |
| 8 | 100% Attane 4404 | 94.2 | 38.7 | 0.79 | 2.14 |
| 9 | 100% Flexomer DFDA 1137 | 95.7 | 36.6 | 1.03 | 2.63 |
| 10 | 30% LE6600/70% Engage 8842 | 72.9 | 18 | 0.17 | 0.42 |
| 11 | 30% HE7541/70% Engage 8842 | 77.3 | 17.0 | 0.24 | 0.62 |
| 12 | 30% HE7541/70% | 95.3 | 44.6 | 2.09 | 5.14 |
| | Flexomer DFDA 1137 | | | | |
| 13 | 30% HE7541/70% Attane 4404 | 96.1 | 44.5 | 2.22 | 5.12 |
| 14 | 80% HE7541/20% Engage 8842 | 97.7 | 51.4 | 4.67 | — |
| 15 | 80% HE7541/20% Attane 4201 | 96.1 | 55.7 | 5.16 | — |
| 16 | 80% HE7541/20% | 97.6 | 55.2 | 5.49 | — |
| | Flexomer DFDA 1137 | | | | |

As seen from FIGS. 3-4, as well as Table 2, the polyolefin blends cover a wide range of durometers and flexural properties to match the polyurethane and polyamide materials such as PEBAX® resins, which are conventionally used in catheters. Additionally, it is evident from FIG. 3 that the polyolefin blends have significantly lower density for a similar durometer PEBAX® material, enabling the catheters to be more flexible and easier to navigate through the vasculature.

### Tubing Examples

PEBAX®, polyolefin resins, and their compounded blends were extruded using a Gimac 25 mm single screw melt extruder. The melt temperatures for the extrusion ranged between 120 to 280° C. across the multiple heat zones in the barrel and die of the extruder. The tubing samples were evaluated for flexural stress on an Instron using a 3-point bend fixture and contact angle

TABLE 3
Flexural Stress and Contact Angle Results for the Tubing Samples

| | | | Wall | Flexural | |
|---|---|---|---|---|---|
| Com- | | | Thick- | Stress @ | Contact |
| position | | OD | ness | 0.5% Strain | Angle |
| # | Tubing Composition | (mm) | (mm) | (MPa) | (°) |
| 1 | 100% Pebax 2533 | 2.21 | 0.27 | 1.58 | 83 |
| 2 | 100% Pebax 5533 | 2.41 | 0.14 | 9.97 | 83 |
| 3 | 100% Pebax 7233 | 2.41 | 0.13 | 27.09 | 85 |
| 4 | 100% Nylon 12 | 2.22 | 0.13 | 41.09 | 87 |
| 5 | 30% LE6600/ | 2.47 | 0.11 | — | 98 |
| | 70% Engage 8842 | | | | |
| 6 | 30% HE7541/ | 2.38 | 0.12 | 4.88 | 104 |
| | 70% Engage 8842 | | | | |
| 7 | 30% HE7541/ | 2.34 | 0.12 | 8.83 | 104 |
| | 70% Flexomer | | | | |
| | DFDA 1137 | | | | |
| 8 | 30% Orevac 18507/70% | 2.37 | 0.12 | — | 95 |
| | Flexomer DFDA 1137 | | | | |
| 9 | 30% HE7541/70% | 2.42 | 0.12 | 7.64 | 98 |
| | Attane 4404 | | | | |
| 10 | 80% HE7541/20% | 2.22 | 0.12 | 18.52 | 99 |
| | Engage 8842 | | | | |
| 11 | 80% HE7541/20% | 2.32 | 0.12 | 17.53 | 93 |
| | Attane 4201 | | | | |
| 12 | 80% HE7541/20% | 2.33 | 0.12 | 16.76 | 87 |
| | Flexomer DFDA 1137 | | | | |
| 13 | 80% Orevac 18507/20% | 2.39 | 0.12 | — | — |
| | Flexomer DFDA 1137 | | | | |
| 14 | 50% HE7541/ | 2.16 | 0.117 | 8.63 | 98 |
| | 50% Engage 8842 | | | | |

### Polyolefin Expanded Tubing—Heat Shrink Example

A polyolefin tubing of composition 11 from Table 2 was extruded with an OD of around 2.67 mm and a wall thickness around 0.18 mm. The tubing was radially expanded by pressurizing the tubing inside out, under heated conditions below the melting peak temperature of the resin. The expanded tubing had an OD of around 3.75 mm and a wall thickness around 0.11 mm. The expanded tubing was recovered over a mandrel measuring 2.92 mm in diameter at a temperature around 100° C.

### Catheter Examples

### Example 1A

A melt extruded HDPE blend tubing as described in US Patent Application Publication No. 2024/0066846, which is incorporated herein by reference in its entirety, was used as a liner in a catheter shaft and braided with a SS flat wire. The catheter shaft was about 36 inches in overall length with the proximal half jacketed in polyolefin tubing of composition 13 from Table 3 and the distal half jacketed in polyolefin tubing of composition 8 from Table 3. The catheter shaft was laminated at 270° C. and tested for trackability using the IDTE3000.

### Example 1B

A dispersion-coated UHMWPE tubing as described in US Patent Application Publication No. 2024/0066186 was used as a liner in a catheter shaft and braided with a SS flat wire. The catheter shaft was about 36 inches in overall length with the proximal half jacketed in polyolefin tubing of composition 13 from Table 3 and the distal half jacketed in polyolefin tubing of composition 8 from Table 3. The catheter shaft was laminated at 290° C. and tested for trackability using the IDTE3000.

### Example 2A

A melt-extruded HDPE blend tubing as described in US Patent Application Publication No. 2024/0066846 was used as a liner in a catheter shaft and braided with a UHMWPE round monofilament. The catheter shaft was about 36 inches in overall length with the proximal half jacketed in polyolefin tubing of composition 13 from Table 3 and the distal half jacketed in polyolefin tubing of composition 8 from Table 3. The catheter shaft was laminated at 290° C. and tested for trackability using the IDTE3000.

### Example 3A

A melt-extruded HDPE blend tubing as described in US Patent Application Publication No. 2024/0066846 was used as a liner in a catheter shaft and braided with a SS flat wire. The catheter shaft was about 24 inches in overall length, jacketed in polyolefin tubing of composition 8 from Table 3. The catheter shaft was laminated at 290° C. and tested on the FTS7000 friction testing system.

### Example 3B

A dispersion-coated UHMWPE tubing as described in US Patent Application Publication No. 2024/0066186 was used as a liner in a catheter shaft and braided with a SS flat wire. The catheter shaft was about 24 inches in overall length, jacketed in polyolefin tubing of composition 8 from Table 3. The catheter shaft was laminated at 290° C. and tested on the FTS7000 friction testing system.

### Example 4A

A melt-extruded HDPE 1 tubing was used as a liner and jacketed in polyolefin tubing of composition 7 from Table 3. The assemblies were laminated at 250° C. and 290° C. and tested for delamination. Cross-section of the 250° C. assembly was also observed under the microscope.

### Example 4B

A dispersion-coated UHMWPE tubing as described in US Patent Application Publication No. 2024/0066186 was used as a liner and jacketed in polyolefin tubing of composition 7 from Table 3. The assembly was laminated at 290° C. and tested for delamination.

### Example 5

A round UHMWPE monofilament with a diameter of 0.127 mm as described in U.S. Patent Application Publication No. 2024/0066777, which is incorporated herein by reference in its entirety, was used to coil around a mandrel to form the first layer of a catheter shaft. A second layer comprising of a 0.08 mm round SS wire covered with a 0.06 mm thick coating of the polyolefin blend of composition 12 from Table 2 was coiled around the UHMWPE coil. The two layered coiled catheter assembly was laminated at 220° C. to form a catheter shaft with the UHMWPE material on the inner layer forming a liner and the polyolefin blend on the SS wire forming a jacket to encompass the SS coil reinforcement.

### Example 6

A round UHMWPE monofilament with a diameter of 0.127 mm as described in U.S. Patent Application Publication No. 2024/0066777 was used to coil around a mandrel to form the first layer of a catheter shaft. A 0.025 mm×0.127 mm round SS wire was coiled around the first layer and a 0.10 mm monofilament of the polyolefin blend of composition 12 from Table 2 was coiled as the third layer in the assembly. The triple layered coiled catheter assembly was laminated at 220° C. to form a catheter shaft with the UHMWPE material on the inner layer forming a liner and the polyolefin blend on the outside forming a jacket to encompass the SS coil reinforcement.

### Example 7

A 0.102 mm×0.254 mm rectangular UHMWPE monofilament as described in U.S. Patent Application Publication No. 2024/0066777 was braided around a mandrel at 60 ppi. The braided structure was reflowed at 220° C. using a heat shrink fusing sleeve to form a composite liner and braid structure.

### Catheter Comparative Examples

### Comparative Example 1

A Zeus manufactured etched PTFE tubing was used as a liner in a catheter shaft and braided with a SS flat wire. The catheter shaft was about 36 inches in overall length with the proximal half jacketed in Pebax 72D and the distal half jacketed in PEBAX® 55D. The catheter shaft was laminated at 250° C. and tested for trackability using the IDTE3000.

### Comparative Example 2

A Zeus manufactured etch PTFE tubing was used as a liner in a catheter shaft and braided with a SS round wire. The catheter shaft was about 36 inches in overall length with the proximal half jacketed in Pebax 72D and the distal half jacketed in PEBAX® 55D. The catheter shaft was laminated at 250° C. and tested for trackability using the IDTE3000.

### Comparative Example 3

A Zeus manufactured etch PTFE tubing was used as a liner in a catheter shaft and braided with a SS flat wire. The catheter shaft was about 24 inches in overall length, jacketed in PEBAX®55D. The catheter shaft was laminated at 250° C. and tested on the FTS7000 friction testing system.

### Comparative Example 4

A Zeus manufactured etch PTFE tubing was used as a liner and jacketed in PEBAX®55D. The assembly was laminated at 250° C. and tested for delamination. The cross-section of the assembly was also observed under the microscope.

TABLE 4
Catheter Constructions

| | Final | | | | | | | | | | |
|---|---|---|---|---|---|---|---|---|---|---|---|
| | Assembly Size | | Liner | | | Reinforcement | | | Jacket | | |
| | ID | OD | | ID | Wall | | Size | | | ID | Wall |
| Example # | mm | | Material | mm | | Material | mm | Type | Material | mm | |
| Example 1A | 1.70 | 2.08 | HDPE | 1.70 | 0.038 | SS | 0.025 × | Braid; | Polyolefin | 2.13 | 0.12 |
| | | | Blend | | | | 0.127 | 80 ppi | Blend | | |
| Example 1B | 1.78 | 2.16 | UHMWPE | 1.80 | 0.038 | SS | 0.025 × | Braid; | Polyolefin | 2.13 | 0.12 |
| | | | | | | | 0.127 | 80 ppi | Blend | | |
| Comparative | 1.68 | 2.11 | PTFE | 1.73 | 0.038 | SS | 0.025 × | Braid; | Pebax | 2.13 | 0.13 |
| Example 1 | | | | | | | 0.127 | 80 ppi | | | |
| Example 2 | 1.70 | 2.01 | HDPE | 1.70 | 0.038 | UHMWPE | 0.064 | Braid; | Polyolefin | 2.13 | 0.12 |
| | | | Blend | | | | | 80 ppi | Blend | | |
| Comparative | 1.68 | 2.11 | PTFE | 1.73 | 0.038 | SS | 0.051 | Braid; | Pebax | 2.13 | 0.13 |
| Example 2 | | | | | | | | 80 ppi | | | |
| Example 3A | 1.65 | 2.06 | HDPE | 1.70 | 0.038 | SS | 0.025 × | Braid; | Polyolefin | 2.13 | 0.12 |
| | | | Blend | | | | 0.127 | 80 ppi | Blend | | |
| Example 3B | 1.63 | 1.96 | UHMWPE | 1.80 | 0.038 | SS | 0.025 × | Braid; | Polyolefin | 2.13 | 0.12 |
| | | | | | | | 0.127 | 80 ppi | Blend | | |
| Comparative | 1.68 | 2.03 | PTFE | 1.73 | 0.038 | SS | 0.025 × | Braid; | Pebax | 2.13 | 0.13 |
| Example 3 | | | | | | | 0.127 | 80 ppi | | | |
| Example 4A - | 1.68 | 2.08 | HDPE | 0.071 | 0.002 | — | — | — | Polyolefin | 2.13 | 0.12 |
| 250 C. | | | | | | | | | Blend | | |
| Example 4A - | 1.68 | 2.08 | HDPE | 0.071 | 0.002 | — | — | — | Polyolefin | 2.13 | 0.12 |
| 290 C. | | | | | | | | | Blend | | |
| Example 4B | 1.80 | 2.13 | UHMWPE | 1.80 | 0.038 | — | — | — | Polyolefin | 2.13 | 0.12 |
| | | | | | | | | | Blend | | |
| Comparative | 1.68 | 2.03 | PTFE | 1.73 | 0.038 | — | — | — | Pebax | 2.13 | 0.13 |
| Example 4 | | | | | | | | | | | |
| Example 5 | 7.77 | 8.31 | UHMWPE | — | — | SS | 0.076 | Coil | Polyolefin | — | — |
| | | | | | | | | | Blend | | |
| Example 6 | 7.77 | 8.20 | UHMWPE | — | — | SS | 0.025 × | Coil | Polyolefin | — | — |
| | | | | | | | 0.127 | | Blend | | |
| Example 7 | 3.45 | 3.86 | UHMWPE | — | — | UHMWPE | N/A | Braid | — | — | — |

TABLE 5
Trackability Data for the Catheters using the IDTE

| | Final | | Catheter Trackability | | Guidewire Trackability | |
|---|---|---|---|---|---|---|
| | Assembly Size | | Maximum | Work of | Maximum | Work of |
| | ID | OD | Force | Advancement | Force | Advancement |
| Example # | mm | | N | N · cm | N | N · cm |
| Example 1A | 1.70 | 2.08 | 1.23 | 10.09 | — | — |
| Example 1B | 1.78 | 2.16 | 1.03 | 8.71 | 0.03 | 0.84 |
| Comparative | 1.68 | 2.11 | 2.08 | 12.97 | 0.03 | 0.78 |
| Example 1 | | | | | | |
| Example 2 | 1.70 | | 0.33 | 4.22 | 0.08 | 3.82 |
| Comparative | 1.68 | | 0.66 | 6.06 | 0.08 | 3.7 |
| Example 2 | | | | | | |

Catheter trackability is a measure of the ease with which a catheter can be advanced through the vasculature. The catheters of the invention comprising polyolefin materials (Examples 1A, 1B and 2) are noted to give lower values of Maximum Force and Work of Advancement relative to comparable catheters constructed with PTFE and PEBAX® (Comp. Examples 1 and 2, respectively). Hence, the catheters of the invention would be expected to navigate the vasculature with greater ease.
Guide Wire trackability is a sensitive measure of the lubricity of the liner material in the final catheter construction. The catheters of the current disclosure can exhibit properties similar to those of catheters comprising PTFE liners.

TABLE 6
Pull Resistance and Lubricity Data for the Catheters

| | Final | | Pull Resistance | | Lubricity | | |
|---|---|---|---|---|---|---|---|
| | Assembly Size | | Maximum | Average | Minimum | Maximum | Average |
| | ID | OD | Force | Force | COF | COF | COF |
| Example # | mm | | N | N | — | — | — |
| Example 3A | 1.65 | 2.06 | 5.92 | 5.33 | 0.83 | 1.21 | 1.05 |
| Example 3B | 1.63 | 1.96 | 6.34 | 5.58 | 0.74 | 1.31 | 1.14 |
| Comparative | 1.68 | 2.03 | 8.14 | 7.51 | 1.02 | 1.67 | 1.54 |
| Example 3 | | | | | | | |

The catheter construction jacketed in the polyolefin materials are more lubricious than the conventional PEBAX® catheters, which would make it easier to traverse them through a tortuous path as they will experience lower resistance.

TABLE 7
Peel Resistance Data for Catheters

| | Final | | | | |
|---|---|---|---|---|---|
| | Assembly | | Peel Force | | |
| | Size | | Maximum | Average | |
| | ID | OD | Force | Force | Failure |
| Example # | mm | | N | | Mode |
| Example | 1.68 | 2.08 | 7.54 | 5.43 | Cohesive |
| 4A - Low | | | | | |
| Temperature | | | | | |
| Example | 1.68 | 2.08 | 6.84 | 5.08 | Cohesive |
| 4A - High | | | | | |
| Temperature | | | | | |
| Example 4B | 1.80 | 2.13 | 5.45 | 3.70 | Cohesive |
| Comparative | 1.68 | 2.03 | 2.30 | 1.95 | Adhesive |
| Example 4 | | | | | |

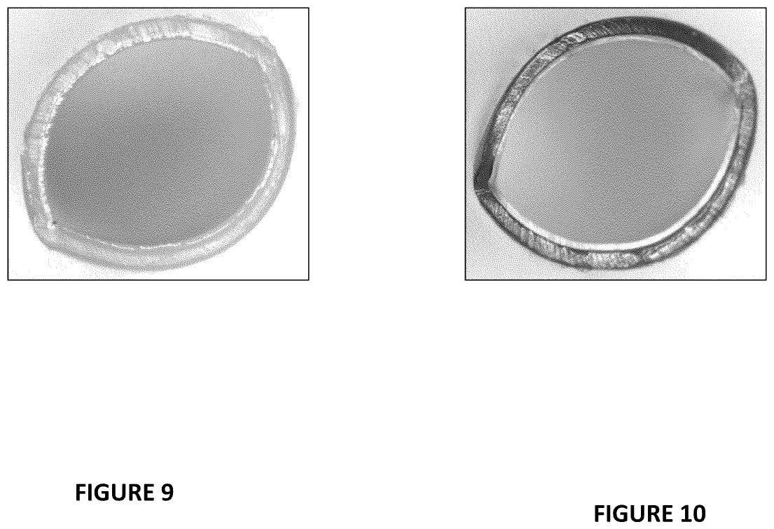

The cross-sectional images of the two catheters as shown in FIGS. 9-10 clearly demonstrate that the conventional PTFE-PEBAX® catheter has two distinct layers, while the polyolefin catheter forms a single layer, reducing the risk of delamination. This also corresponds to the peel force data wherein the polyolefin catheters undergo a cohesive failure at a force value two to three times that of a conventional PTFE-PEBAX® catheter, which usually undergoes an adhesive failure. Additionally, the liners in the polyolefin catheter assemblies were not surface treated or coated with a tie-layer and underwent cohesive failure.

Many modifications and other embodiments of the invention will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A catheter with a proximal end and a distal end defining a length, L, comprising:
a polyolefin-based liner forming an inner surface of the catheter for at least a part of L;
a reinforcing braid disposed in and/or around the polyolefin liner for at least a part of L; and
a polyolefin-based jacket forming an outer surface of the catheter, disposed in and/or around the braid for at least a part of L.

2. The catheter of claim 1, wherein the polyolefin-based liner forms the inner surface of the catheter for the full length of the catheter.

3. The catheter of claim 1, wherein the polyolefin-based jacket forms the outer surface of the catheter for the full length of the catheter.

4. The catheter of claim 1, wherein the polyolefin-based liner forms the inner surface of the catheter for less than the full length of the catheter.

5. The catheter of claim 1, wherein the polyolefin-based jacket forms the outer surface of the catheter for less than the full length of the catheter.

6. The catheter of claim 1, wherein the polyolefin-based liner consists essentially of one or more polyolefins.

7. The catheter of claim 1, wherein the polyolefin-based jacket consists essentially of one or more polyolefins.

8. The catheter of claim 1, wherein one or both of the polyolefin-based liner and the polyolefin-based jacket comprises one or more additives.

9. The catheter of claim 1, wherein the polyolefin-based liner has a density that differs from the density of the polyolefin-based jacket.

10. The catheter of claim 9, wherein the polyolefin-based liner comprises one or more polyolefin resins that have a density between 0.90 g/cm³ and 0.97 g/cm³.

11. The catheter of claim 9, wherein the polyolefin-based jacket comprises one or more polyolefin resins that have a density between 0.85 g/cm³ and 0.96 g/cm³.

12. The catheter of claim 9, wherein the density of the polyolefin-based jacket is less than or equal to the density of the polyolefin-based liner.

13. The catheter of claim 1, wherein the polyolefin-based liner comprises LDPE, LLDPE, HDPE, HMWPE, or UHMWPE or a blend of any two or more thereof.

14. The catheter of claim 1, wherein the polyolefin-based liner has an average wall thickness of less than 0.200 mm.

15. The catheter of claim 1, wherein the polyolefin-based jacket comprises VLDPE, ULDPE, LDPE, LLDPE, HDPE, HMWPE, or UHMWPE or a blend of any two or more thereof.

16. The catheter of claim 1, wherein the polyolefin-based liner comprises a blend of one or more polyethylene homopolymers and one or more polyethylene copolymers.

17. The catheter of claim 1, wherein the reinforcing braid comprises stainless steel or nitinol wires.

18. The catheter of claim 1, wherein the reinforcing braid comprises a liquid crystal polymer, polypropylene or UHMWPE monofilament, multifilament or yarn.

19. The catheter of claim 1, wherein the polyolefin-based jacket comprises a blend of one or more polyethylene homopolymers, one or more polyethylene copolymers, and one or more ethylene-propylene copolymers.

20. The catheter of claim 1, comprising two or more polyolefin-based jackets, each having a different density and each disposed in and around the braid for at least less than L, wherein the two or more polyolefin-based jackets are joined together.
